(19) United States
(12) Patent Application Publication
(10) Pub. No.: US 2025/0060348 A1
(43) Pub. Date: Feb. 20, 2025

(54) WIRELESS LC-RESONANCE SENSOR SYSTEM FOR MONITORING TEMPERATURE, PRESSURE AND GAS SPECIES INSIDE AN ENCLOSURE

(71) Applicant: X-wave Innovations, Inc., Gaithersburg, MD (US)

(72) Inventors: Dan Xiang, Gaithersburg, MD (US); DHEERAJ VELICHETI, Gaithersburg, VA (US); Adam HARWOOD, Gaithersburg, VA (US)

(73) Assignee: X-wave Innovations, Inc., Gaithersburg, MD (US)

(21) Appl. No.: 18/770,626

(22) Filed: Jul. 11, 2024

Related U.S. Application Data
(60) Provisional application No. 63/526,087, filed on Jul. 11, 2023.

(51) Int. Cl. G01K 7/34; G01L 9/10; G01L 9/12

(52) U.S. Cl. CPC G01N 33/0068; G01K 7/34; G01L 9/10; G01L 9/12

(57) ABSTRACT

The embodiments disclose an LC-resonance sensor system for monitoring internal conditions of an enclosure. The system includes an LC temperature sensor, an LC pressure sensor, and an LC gas species sensor that each have an inductor and capacitor. The system uses a transmitting coil that transmits a wireless signal to each of the LC sensors, and receiving coils for each of the sensors that receive wireless signals from the LC sensors.

### STATEMENT REGARDING FEDERALLY SPONSORED RESEARCH OR DEVELOPMENT

This invention was made with government support under DE-SC0022827 awarded by the U.S. Department of Energy. The government has certain rights in the invention.

### CROSS-REFERENCE TO RELATED APPLICATIONS

Not applicable.

### FIELD OF INVENTION

The present embodiments are directed to the technical field of sensor and sensor systems. More particularly, the present embodiments relate to the field of electromagnetic resonant circuit (LC-resonator) sensors used to monitor the internal conditions inside a sealed enclosure using wireless means of interrogation through a wall to track the temperature, pressure and gas composition inside of the enclosure.

### BACKGROUND

In situations involving condition monitoring and wireless sensing, it is not uncommon to face challenges related to powering and communicating with sensors that are enclosed in sealed metal containers, vacuum or pressure vessels, or isolated by metal walls, such as vessel hulls, bulkheads, aircraft and spacecraft fuselages, and vehicle armor. While batteries are a common power source for sensors, they may not always be suitable due to installation environment, volume, or other limitations. In addition, the complexity and cost of replacing batteries in these enclosures can be problematic. Historically, metal wall penetrations have been employed to convey wires between a sensor inside the enclosure and a receiver outside the enclosure. Nevertheless, there are various practical design considerations that must be addressed when utilizing wires to transmit power and data through a metallic structure. These include the potential for toxic chemical leakage, pressure or vacuum loss, and complications in managing thermal and electrical insulation. Additionally, introducing wires through a metallic structure's wall can compromise its strength and integrity and subject it to a heightened risk of cracking due to stress fatigue. Furthermore, this practice can also escalate the overall lifetime maintenance costs.

In modern aeronautical and aerospace fields, dependable sensors that can be powered and operated wirelessly are crucial, particularly for maintenance and monitoring purposes. For instance, during NASA's Mars Sample Return Mission, wireless sensors will be utilized on the sealed sample container to detect pressure leaks and prevent potential contamination. In this scenario, power and data must be transmitted through the metal container wall without any penetration. Similarly, there is a pressing need for a solution that enables successful transmission of power and data through metal walls for sensors embedded in conductive materials without physical penetration to facilitate wireless sensing and health monitoring of aircraft. These two situations are similar to the situation of monitoring the contents of a dry storage canister filled with spend nuclear fuel.

Spent nuclear fuel (SNF) removed from a nuclear reactor can be stored in a dry storage canister (DSC). As more and more of the nuclear industry's SNF is placed into dry nuclear canister systems (≈300 per year), concern associated with the safe storage of the SNF before its final disposal has been growing. Monitoring internal conditions of SNF-DSC systems to identify or predict fuel cladding failure and fuel assembly structural degradation or corrosion is crucial for regulatory organizations and public safety. The attributes to be monitored in a DSC include helium leakage, internal pressure, temperature profiles, gas composition, xenon or krypton gas release, radiation dose levels, etc.

### SUMMARY

The problems and shortcomings of traditional devices are overcome by the embodiments for a wireless LC-resonance sensor system for monitoring temperature, pressure, and gas species that can be used to measure parameters inside an enclosure. In an embodiment, these sensors have to be self-powered or wirelessly powered through a metal wall. The signals from the sensors also need to be wirelessly transmitted. Additionally, when electromagnetic means of wireless communication are used, the metal wall tend to shield such signals. On top of these challenges, the sensors for a DSC are required to operate for long-term (>10 years) in high-temperature (100-200 C) and high-radiation (gamma and neutron) environment.

In one aspect, an LC-resonance sensor system for monitoring internal conditions of an enclosure can include an LC temperature sensor comprising an inductor and capacitor, an LC pressure sensor comprising an inductor and capacitor, an LC gas species sensor comprising an inductor and capacitor, a transmitting coil that transmits a wireless signal to each of the LC temperature sensor, LC pressure sensor, and LC gas species sensor; and a first receiving coil for receiving wireless signals from the LC temperature sensor, a second receiving coil for receiving wireless signals from the LC pressure sensor, and a third receiving coil for receiving wireless signals from the LC gas species sensor.

### BRIEF DESCRIPTION OF THE DRAWINGS

The present embodiments are better understood by reference to the following detailed description when considered in connection with the accompanying drawing, wherein:

FIG. 1(A) shows an illustration of exemplary LC-sensors installed inside a DSC unit and wirelessly interrogated from outside the canister, through the steel wall, using a transmitting (Tx) coil and receiver (Rx) coil;

FIG. 1(B) shows a graphic with signals expected across each of the three inductors of the embodiments;

FIG. 2 is a schematic of transmission side electronics used to drive large currents into a Tx coil of the embodiments;

FIG. 3 is a schematic of receiver side electronics of the embodiments;

FIG. 4(A) shows an illustration of the configuration of the LC-resonance sensor system for internal condition monitoring with three LC-sensors inside the DSC unit of the embodiments;

FIG. 4(B) is an exemplary plot of signals from LC temperature sensor in the time domain;

FIG. 4(C) is an exemplary plot of signals from LC gas species sensor in the time domain;

FIG. 4(D) is an exemplary plot of signals from LC pressure sensor in the time domain;

FIG. 5 shows a temperature sensitive inductor used in an LC temperature sensor of the embodiments;

FIG. 6 shows a flowchart with the various stages involved in the processing of the measured signal at the LC temperature sensor receiver (Rx) coil for estimation of the temperature inside the DSC canister;

FIG. 7 (A) shows an exemplary plot of the change in peak-to-peak amplitude and of the signal from the temperature sensor;

FIG. 7 (B) shows an exemplary plot of the change in frequency of the signal from the temperature sensor;

FIG. 8 is an exemplary plot that compares the temperature measured based on peak-to-peak amplitude change at the LC-sensor to the data obtained using a thermocouple;

FIG. 9 shows a schematic of a two piezo-diaphragm based pressure sensor without the inductor of the embodiments;

FIG. 10 shows a flowchart with the various stages involved in the processing of the measured signal at the LC pressure sensor receiver (Rx) coil for estimation of the pressure inside the DSC canister; FIG. 11 (A) is an exemplary plot of signals from the pressure sensor in frequency domain at different pressure levels;

FIG. 11 (B) is an exemplary plot that shows a normalized shift of the frequency peaks with pressures of the signals plotted in FIG. 9(A);

FIG. 12 is an exemplary plot that compares the pressure levels measured based on shifts in frequency peaks of the signal from the LC-sensor to the data obtained from a pressure gauge;

FIG. 13 shows a schematic of the acoustic-resonator gas species sensor with a piezo-diaphragm at one end of cylindrical tube with the other end sealed off by a reflector plate without the inductor of the embodiments;

FIG. 14 shows a flowchart with the various stages involved in the processing of the measured signal at the LC gas species sensor receiver (Rx) coil for estimation of the gas species content inside the DSC canister;

FIG. 15 (A) in an exemplary plot of signals from the gas species sensor in frequency domain for air and helium environment;

FIG. 15 (B) is an exemplary plot that shows shift the frequency peaks with changing gas type of the signals of FIG. 12 (A); and

FIG. 16 is an exemplary schematic that compares the gas type estimated by tracking shift in frequency peaks of the signal from the LC-sensor to the actual case.

### DETAILED DESCRIPTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims. The present disclosure is to be considered as an exemplification of the invention and is not intended to limit the invention to the specific embodiments illustrated by the figures or description herein.

Various embodiments of the present invention may incorporate one or more of these and the other features described herein. The following detailed description taken in conjunction with the accompanying drawings may provide a better understanding of the nature and advantages of the present invention. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications. While the invention has been described with a certain degree of particularity, it is manifest that many changes may be made in detail of construction and the arrangement of components without departing from the spirit and scope of this disclosure. The present disclosure is to be considered as an exemplification of the principles of the invention and is not intended to limit the invention to the specific embodiments illustrated herein by the figures or description above.

Conventionally, various solutions for transmitting power and data through metal walls without intrusive procedures such as drilling holes have been proposed. On a broad level these methods can be classified in two groups: electromagnetic and acoustic/ultrasonic. Electromagnetic coupling-based techniques, which include inductive coupling, capacitive coupling, and magnetic resonance coupling, are one such category of solutions. Electromagnetic-based methods can achieve some level of power and data transmission through metal walls, but their effectiveness is hindered by the strong Faraday shielding effect or skin effect exhibited by ferromagnetic metallic barriers or thick non-ferromagnetic metallic barriers, rendering them highly inefficient and impractical. Therefore, the frequency of inspection has to kept below 5 kHz such that EM waves can penetrate the one-half inch thick stainless steel wall of a DSC unit.

Resonant circuit (LC-resonator) sensors can be used for such cases. However, measuring the resonance at the LC circuit through a metal wall using only one coil excited at multiple frequencies does not provide a strong signal which could be used for monitoring the internal conditions inside a canister. In the embodiments, a three-coil system offers a superior design to interrogate the LC-sensor inside a metal enclosure, where a transmission coil transmits energy into the LC circuit through the wall, and a separate reception coil picks up the ringing from the LC circuit. Using such an exemplary design, no multifrequency excitation is required.

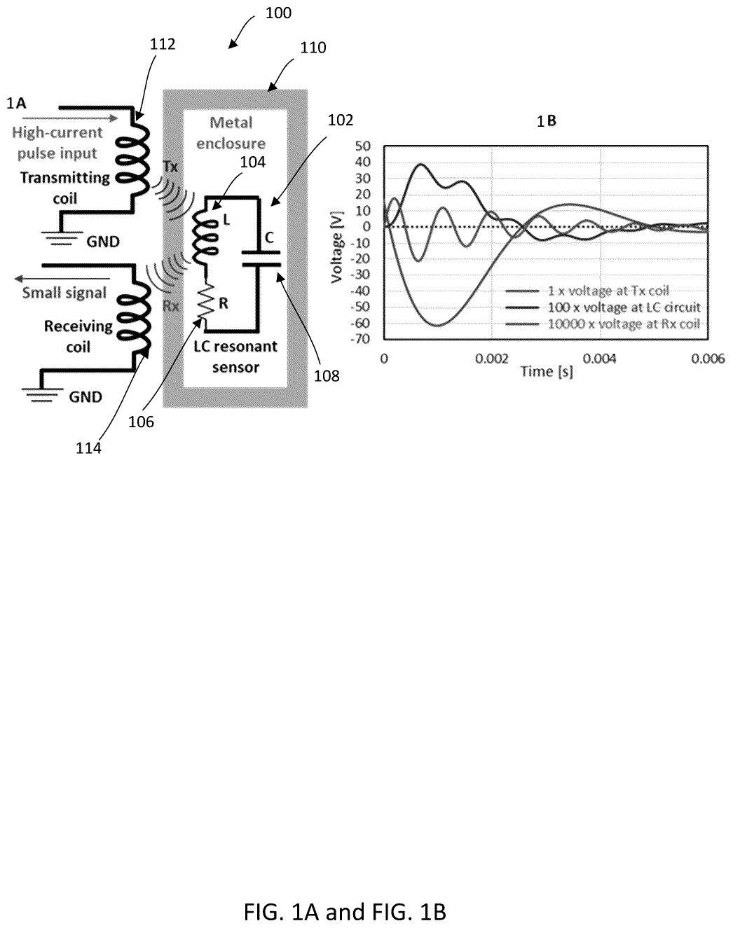

Referring to FIGS. 1-13, embodiments of the invention are illustrated. The embodiments may be employed for long term (e.g., more than 10 years) monitoring of the internal conditions such as temperature, pressure and gas composition inside an enclosed unit, such as a SNF-DSC system stored inside concrete overpack structures. FIG. 1(A) shows an illustration of the configuration of an exemplary LC-resonance sensor system 100. The system comprises an LC sensor 102 that includes an inductor 104, resistor 106, and capacitor 108. The LC-sensor 102 can be installed inside a DSC unit 110 and wirelessly interrogated from outside the canister, through the steel wall, using a transmitting (Tx) coil 112 and receiver (Rx) coil 114. FIG. 1(B) is a plot of the transmission signal, the voltage at the LC-sensor 102 multiplied by a factor of 100, and the voltage the Rx coil 114 multiplied by a factor of 10000 to highlight the strength of the output when compared to the input signal. Therefore, electronics are required to transmit large currents into the Tx coil 112 and amplify the small signals sensed at the Rx coil 114.

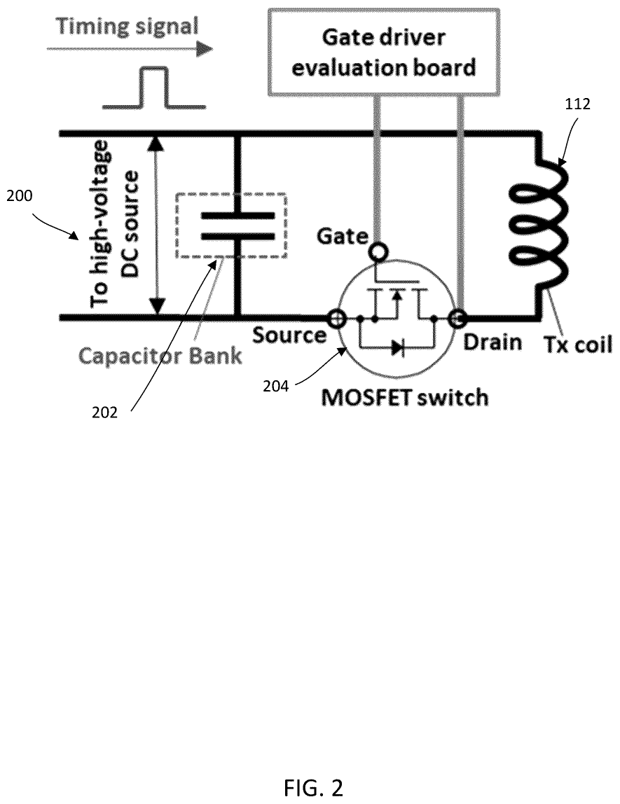

FIG. 2 is a schematic of transmission side electronics used to drive large currents into a Tx coil of the embodiments. The transmission side includes a circuit 200 in series with a high-voltage DC source, a capacitor bank 202, and a MOSFET switch 204 which can be used to drive large currents into the Tx coil.

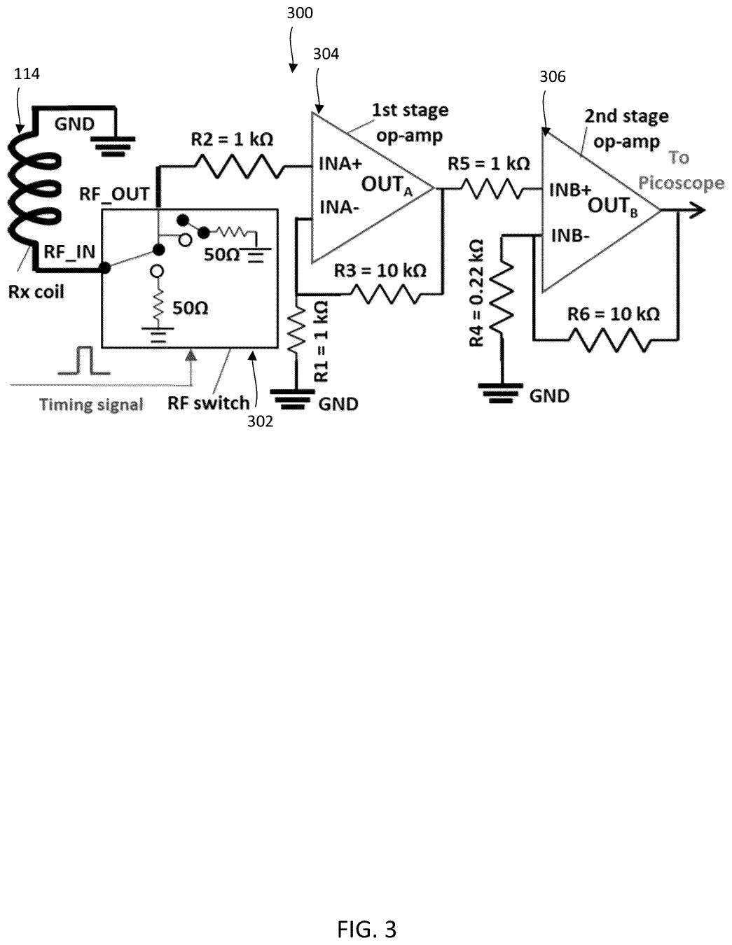

FIG. 3 is a schematic of receiver side electronics of the embodiments where the Rx coil 114 receives signals from an LC sensor. The receiver side circuit 300 includes an RF switch 302 to disconnect the preamplifier 304 from the Rx coil 114 during transmission, and a 58 dB-gain low-noise two-stage preamplifier 306. All specifications in FIG. 3 are exemplary and may vary depending upon the final application of the embodiments. An output of the receiver side circuit 300 can flow to a picoscope (not shown).

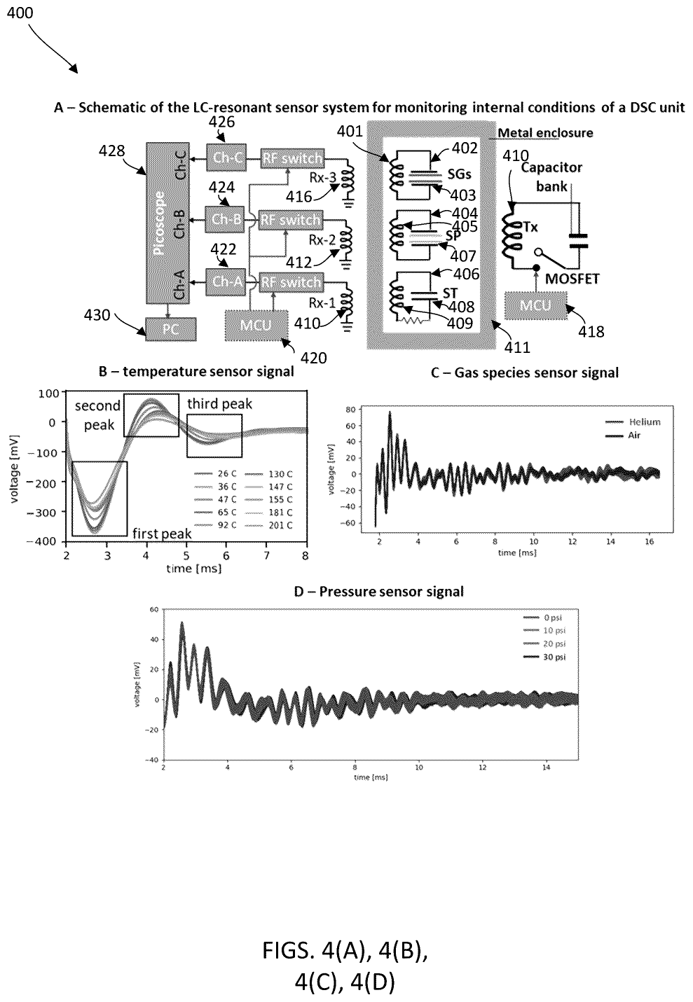

FIG. 4(A) shows an illustration of the configuration of the LC-resonance sensor system for internal condition monitoring with three LC-sensors inside the DSC unit of the embodiments. An exemplary LC-resonance sensor system 400 for internal condition monitoring of a DSC unit can include three exemplary LC-sensors 402 for gas species, 404 for pressure, and 406 for temperature installed inside the DSC unit 411 and wirelessly interrogated from outside the canister 411 using a single transmitting (Tx) coil 410 on the transmission side and three receiver (Rx) coils 412, 414, 416 on the receiver side. Signals through transmitter coil 410 can be controlled by controller processor 418, and signals received on the receiver side can be controlled by controller processor 420. Signals from LC-sensor for temperature 406 can be received by Rx receiver coil 410 and transmitted through a separate channel 422 to picoscope 428. Signals from LC-sensor for pressure 404 can be received by Rx receiver coil 412 and transmitted through a separate channel 424 to picoscope 428. Signals from LC-sensor for gas species 402 can be receive by Rx receiver coil 416 and transmitted through a separate channel 426 to picoscope 428. All signals received by the picoscope 428 can be transmitted to a computer processor 430, which has a memory, for executing signal processing algorithms to further analyze the signals.

The present embodiments for an electromagnetic sensor system can contain three resonant circuit (LC-resonator) sensors 402, 404, 406, an interrogator unit (transmitting coil 410 and receiving coils 410, 412, 416), high-current transmitting circuit 200, high-gain low-noise receiving amplifier 304, signal processing algorithms in processors with memory in controllers 420 and 418 and/or within application software on computer processor 430 with a graphic user interface (GUI). LC gas sensor 402 can include an inductor (L) 401 and capacitor (C) 403 connected together and are installed inside the DSC canister 411. LC pressure sensor 404 can include an inductor (L) 405 and capacitor (C) 407 connected together and are installed inside the DSC canister 411. LC temperature sensor 406 can include an inductor (L) 409 and capacitor (C) 408 connected together and are installed inside the DSC canister 411.

The transmitting coil 410 can be placed outside the DSC canister 411, when injected with a high-current pulse, generates a large time-varying magnetic field penetrating the DSC canister 432 metal wall. The transmitted magnetic field induces a voltage at the inductors of the resonant circuits of the LC-sensors, wirelessly powering the resonant circuits 402, 404, 406, resulting in the ringing of the resonant circuits. The ringing of the resonant circuits generates a time-varying magnetic field which penetrates through the metal canister 432 wall and induces a voltage at the receiver coil 410, 412, 416 placed outside the canister 432.

FIG. 4(B) is an exemplary plot of signals from LC temperature sensor 406 in the time domain. FIG. 4(C) is an exemplary plot of signals from LC gas species sensor 402 in the time domain. FIG. 4(D) is an exemplary plot of signals from LC pressure sensor 404 in the time domain.

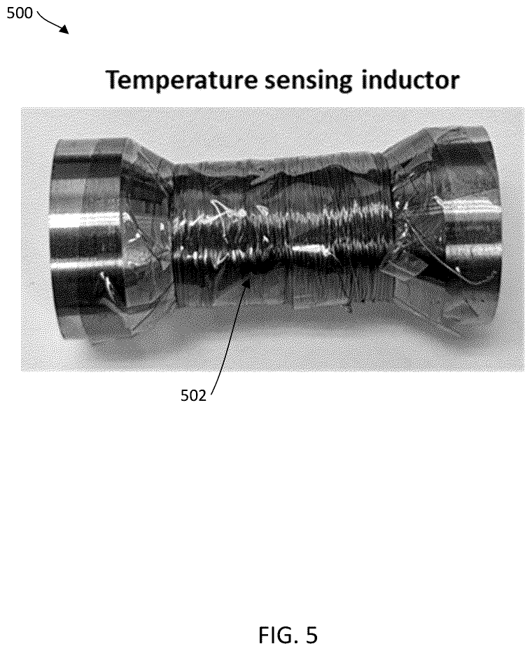

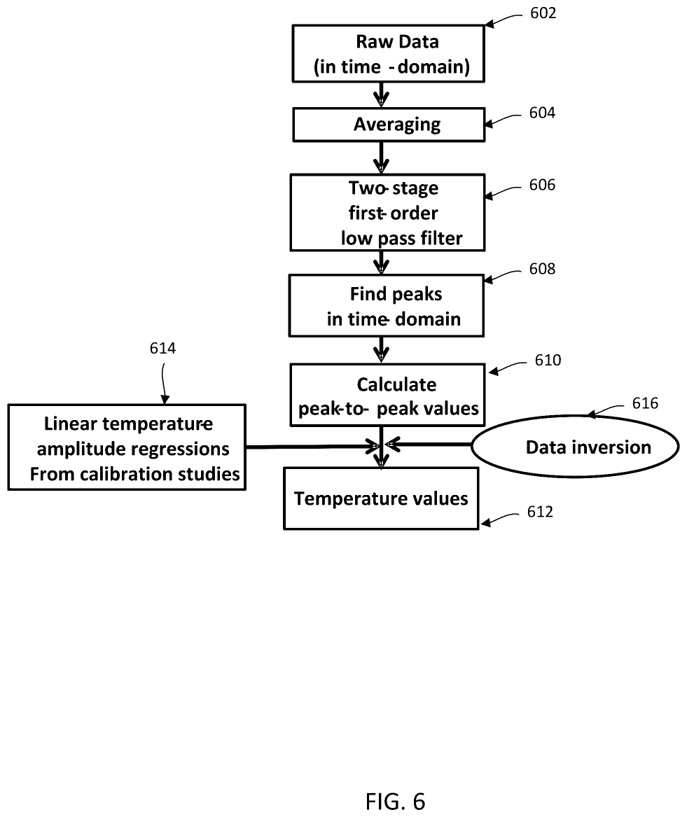

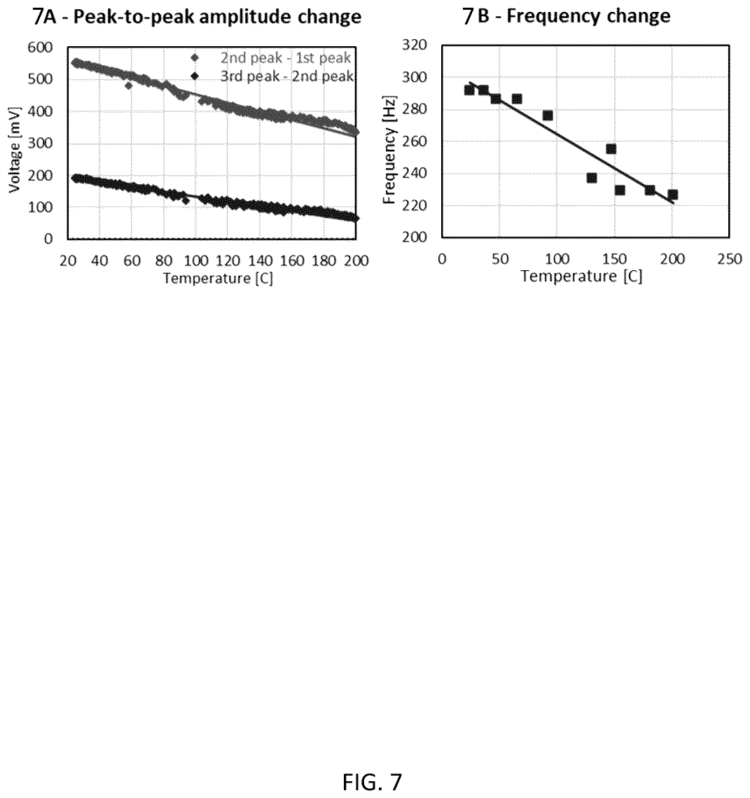

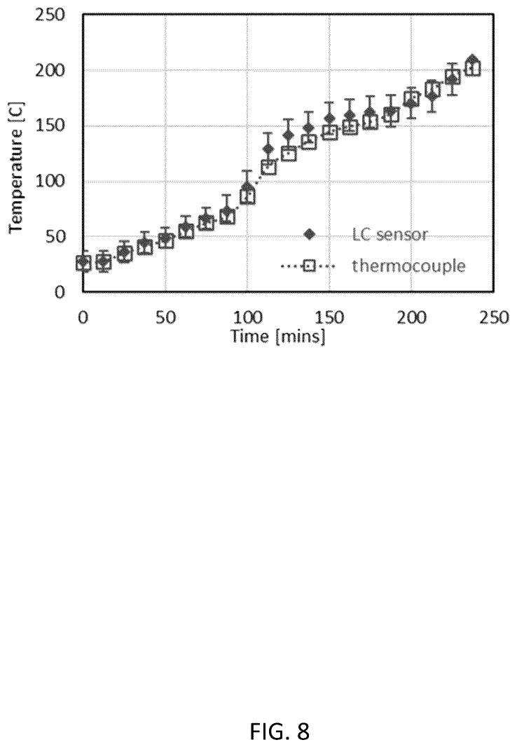

FIG. 5 shows a temperature sensitive inductor 500 used in an LC temperature sensor 406 of the embodiments. The LC-resonant temperature sensor 406 includes a capacitor and an inductor 500 with coil windings 502 with resistance that is sensitive to temperature. Change in the resistance at the inductor 500 causes the frequency and amplitude of the ringing at this sensor 406 to change with temperature, which is exploited to track temperature inside the canister 432. FIG. 6 shows an exemplary flowchart of the embodiments highlighting the different steps involved in the processing of signals from the LC temperature sensor by the computer processor and signal processing algorithms. First, the n number of waveforms recorded as a part of a dataset 602 are averaged 604 into one waveform. Second, each averaged waveform is put through two stages of a first-order low-pass filter 606, such as Butterworth filters. For the exemplary plot of signals from LC temperature sensor, the cutoff frequency for the first stage was set at 10 kHz and 5 kHz for the second stage. Third, the peakdetect function within the peakdetect module in python is used to identify the peaks 608 on the averaged and filtered waveform. Fourth, the peak-to-peak voltages are calculated 610, and these values are then used to calculate the temperature 612 inside the metal enclosure using the linear regression trends 614 and in some embodiments data inversion 616 established for the temperature sensor during the calibration studies. FIG. 7 (A) shows an exemplary plot of the change in peak-to-peak amplitude and of the signal from the temperature sensor. FIG. 7 (B) shows an exemplary plot of the change in frequency of the signal from the LC temperature sensor 406. FIG. 8 is an exemplary plot that compares the temperature measured based on peak-to-peak amplitude change at the LC temperature sensor 406 to the data obtained using a thermocouple.

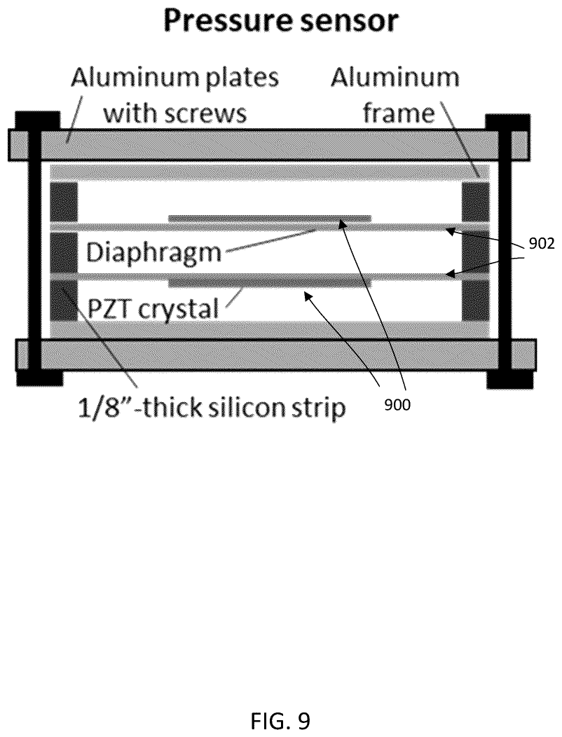

FIG. 9 shows a schematic of a two piezo-diaphragm based pressure sensor 404 without the inductor for the LC pressure sensor 404, which forms the capacitive part of the LC pressure sensor 404 of the embodiments. The LC-resonant pressure sensor 404 includes an inductor 405, and piezo crystal 900 for capacitor 407. The piezo crystal 900 is bonded to a circular diaphragm 902 forming an acoustic diaphragm which mechanically self-resonates at a frequency controlled by the geometric parameters of the diaphragm and the density of the medium surrounding the diaphragm. Therefore, changes in pressure or gas content inside DSC 411 causes the density to change, which in turn changes the frequency response of the piezo-diaphragm. Such change in the frequency response at the piezo-diaphragm changes the frequency of the ringing at the LC-resonant pressure circuit and is utilized to track the density of the gas inside the canister 411.

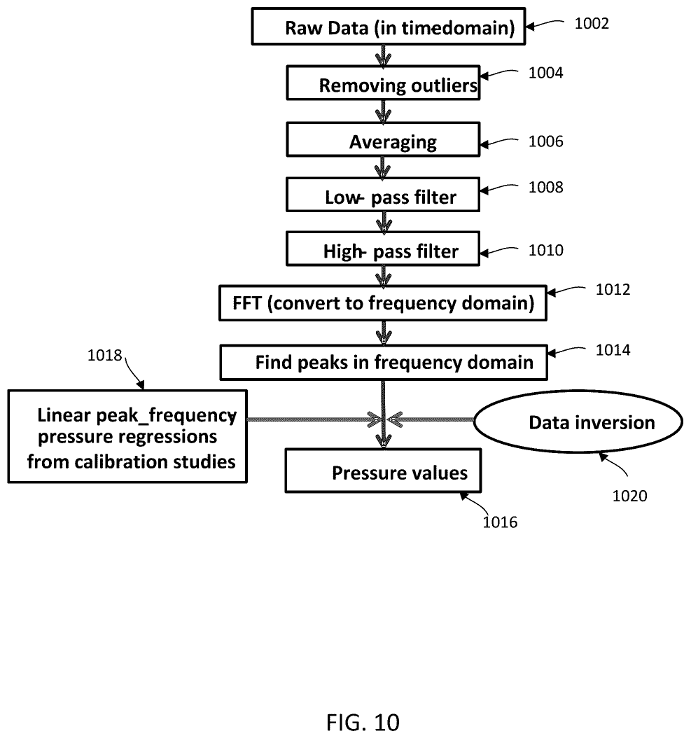

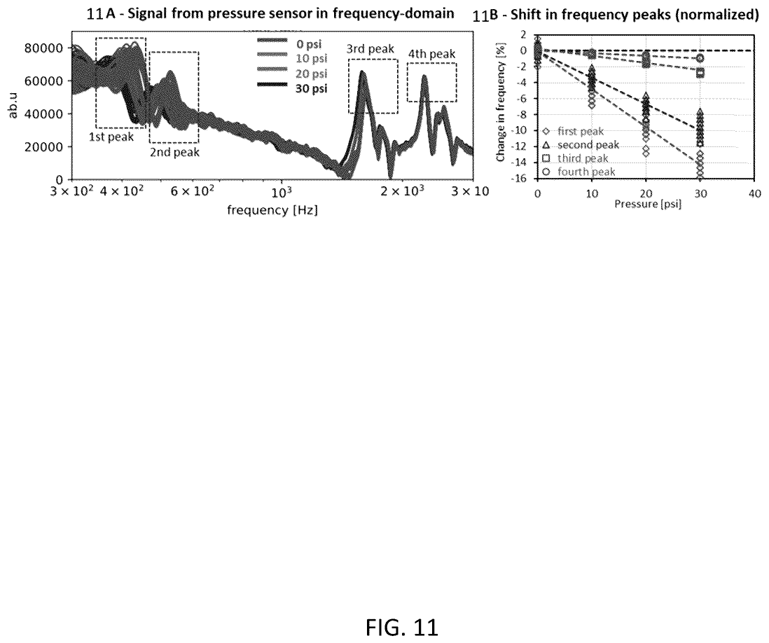

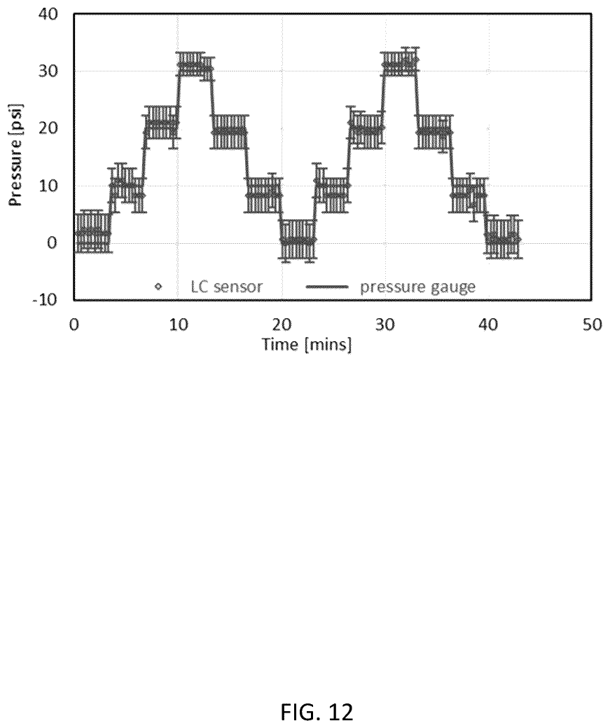

FIG. 10 shows an exemplary flowchart of the embodiments highlighting the different steps involved in the processing of signals from the LC pressure sensor by the computer processor using signal processing algorithms. First, the n number of waveforms recorded for each dataset 1002 are first processed for outliers 1004 by assigning an error value to each waveform. This error value is the sum of error calculated at each sample point, which in turn is the square of the difference between the value and median value obtained from n waveforms. Next, waveforms with error values higher than 125% of the lowest value are rejected and rest of the waveforms are then averaged into a single waveform 1006. Second, the averaged waveforms are first put through a first-order filter 1008, such as Butterworth low-pass filter. For the exemplary plot of signals from LC pressure sensor, the cutoff frequency was set at 5 kHz followed by a first-order high-pass filter 1010 with a cut-off frequency at 1 KHz. Third, a FFT 1012 of the averaged and filtered waveform is obtained which represents the filtered signal in frequency domain. Fourth, the peak detection function is used on the averaged and filtered pressure sensor signal in the frequency domain 1014. Fifth, the frequency value at which the two peaks occur are used to calculate the pressure levels 1016 using the linear peak frequency-pressure relationship 1018 established during the calibration studies. FIG. 11 (A) is an exemplary plot of signals from the pressure sensor in frequency domain at different pressure levels. FIG. 11 (B) is an exemplary plot that shows a normalized shift of the frequency peaks with pressures of the signals plotted in FIG. 11(A). FIG. 12 is an exemplary plot that compares the pressure levels measured based on shifts in frequency peaks of the signal from the LC-sensor to the data obtained from a pressure gauge.

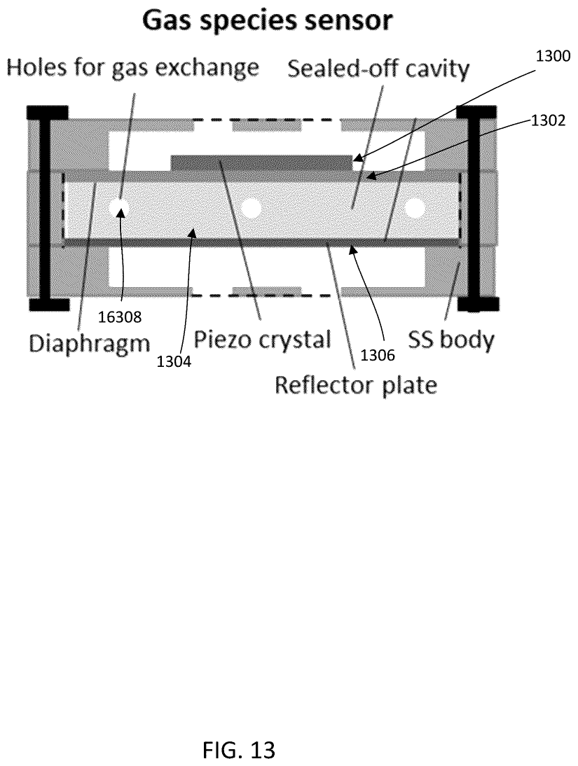

FIG. 13 shows a schematic of the acoustic-resonator gas species sensor 402 with a piezo-diaphragm at one end of cylindrical tube with the other end sealed off by a reflector plate without the inductor of the embodiments. In FIG. 13, the acoustic resonator based gas species sensor 402 can include a piezo-diaphragm comprising a piezo crystal 1300 and diaphragm 1302 at one end of cylindrical tube 1304 with the other end sealed off by a reflector plate 1306, which forms the capacitive part of the LC gas species sensor 402. In the embodiments, the piezo crystal 1300 is bonded to a circular diaphragm 1302 attached to one end of a cylindrical metal tube 1304 with the other end of the tube closed using a metal plate 1306. Small holes 1308 can be fabricated on the sides of the metal tube to enable the exchange of gases. The acoustic diaphragm 1302 along with the metal tube 1304 closed on one end with a rigid plate 1306 forms an acoustic resonator which mechanically self-resonates at a frequency controlled by the geometric parameters of the diaphragm 1302, the density and speed of sound in the medium surrounding the diaphragm 1302. Therefore, changes in gas content inside DSC 411 causes the density and speed of sound to change, which in turn changes the frequency response of the acoustic resonator. Such change in the frequency response at the acoustic resonator changes the frequency of the ringing at the LC-resonant gas species sensor 1302 and is utilized to track the composition of the gas inside the canister 411. It should be noted that the LC gas species sensor 402 is sensitive to both density and speed of sound change in the medium, therefore, the effect of change in density has to be compensated to obtain the speed of sound data which is related to the average molar mass of the gaseous medium inside the DSC 411.

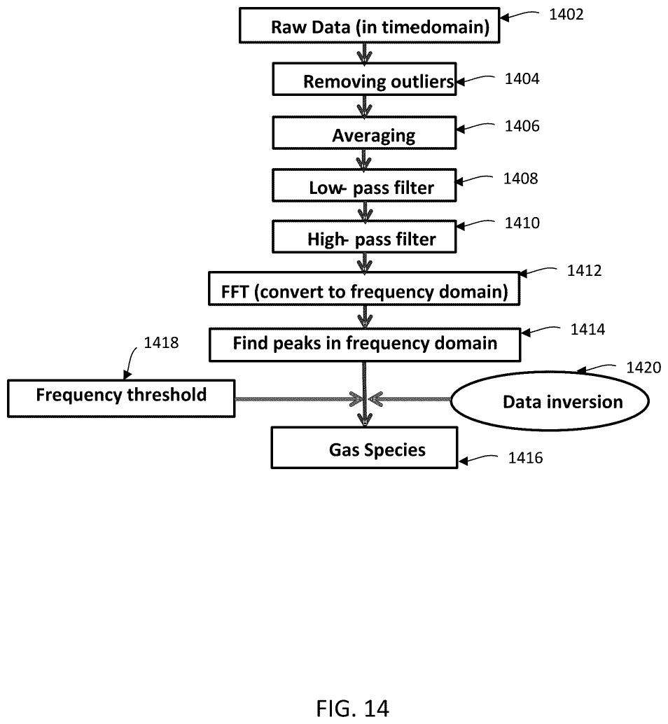

FIG. 14 shows an exemplary flowchart of the embodiments highlighting the different steps involved in the processing of signals from the LC gas species sensor by the computer processor using signal processing algorithms. Due to the similarity for the sensor type, the signal processing for LC gas species sensor is similar to the LC pressure sensor with two differences. First, for the exemplary plot of signals from LC gas species sensor, the cutoff frequencies for the low-pass and high-pass filters are set at 100 Hz and 700 Hz respectively. Second, unlike the pressure sensor data, once the two peaks in frequency domain are obtained, the sum of these two frequencies is used to estimate the gas species inside the metal enclosure/test chamber. FIG. 14 shows an exemplary flowchart of the embodiments highlighting the different steps involved in the processing of signals by the computer processor using signal processing algorithms from the LC pressure sensor. First, the n number of waveforms recorded for each dataset 1402 are first processed for outliers 1404 by assigning an error value to each waveform. This error value is the sum of error calculated at each sample point, which in turn is the square of the difference between the value and median value obtained from n waveforms. Next, waveforms with error values higher than 125% of the lowest value are rejected and rest of the waveforms are then averaged into a single waveform 1406. Second, the averaged waveforms are first put through a first-order filter 1408, such as Butterworth low-pass filter. For the exemplary plot of signals from LC pressure sensor, the cutoff frequency was set at 5 kHz followed by a first-order high-pass filter 1410 with a cut-off frequency at 1 KHz. Third, a FFT 1412 of the averaged and filtered waveform is obtained which represents the filtered signal in frequency domain. Fourth, the peak detection function is used on the averaged and filtered pressure sensor signal in the frequency domain 1414. Fifth, the frequency value at which the two peaks occur are used to calculate the pressure levels 1416 using the linear peak frequency-pressure relationship 1418 established during the calibration studies.

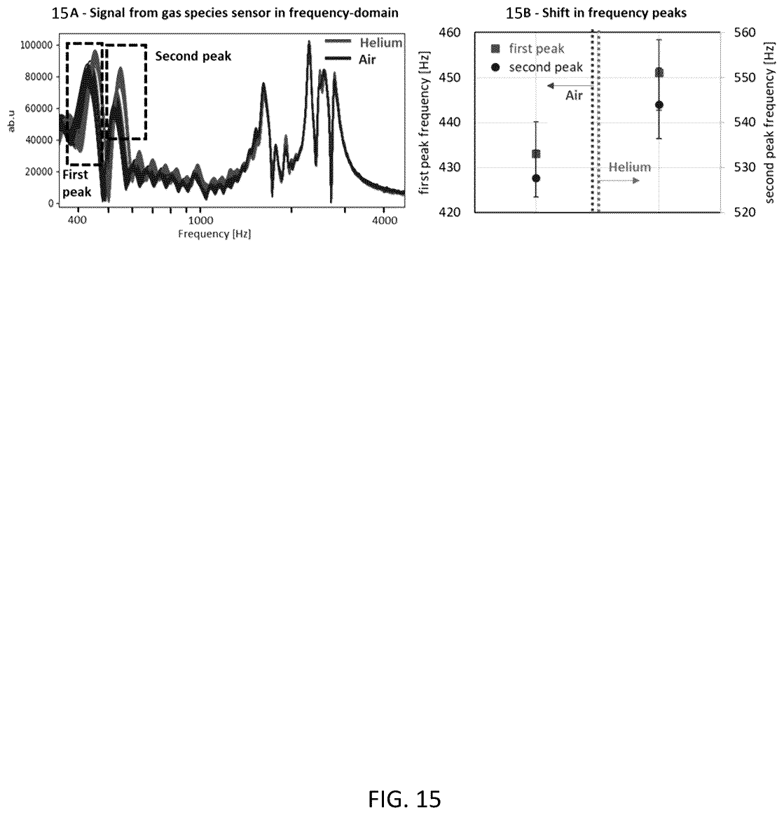

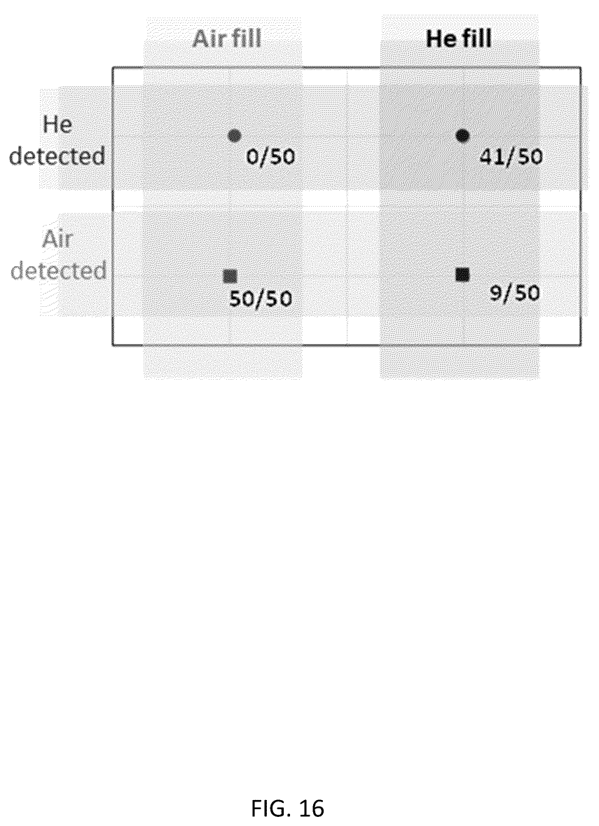

FIG. 15 (A) shows the signals from the LC gas species sensor 402 in frequency domain for air and helium environment, and FIG. 15 (B) shows the shift of the frequency peaks with changing gas type. These changes are due to changes in the density and speed of sound in the medium. Therefore, when measuring both pressure and gas species simultaneously, the density estimated by the LC-pressure sensor 404 is used along with the LC-gas species sensor 402 data to estimate the average molar mass of the gas inside the DSC unit 411. Consequently, the estimated average molar mass can then be used to estimate the pressure levels from the density values measured by the LC pressure sensor 404. FIG. 16 compares the gas type estimated by tracking shift in frequency peaks of the signal from the LC gas species 402 sensor to the actual case.

While the invention has been described with a certain degree of particularity, it is manifest that many changes may be made in detail of construction and the arrangement of components without departing from the spirit and scope of this disclosure. The present embodiment is to be considered as an exemplification of the invention and is not intended to limit the invention to the specific embodiments herein illustrated by the figures or description above.

What is claimed is:

1. An LC-resonance sensor system for monitoring internal conditions of an enclosure, comprising:
an LC temperature sensor comprising an inductor and capacitor;
an LC pressure sensor comprising an inductor and capacitor;
an LC gas species sensor comprising an inductor and capacitor;
a transmitting coil that transmits a wireless signal to each of the LC temperature sensor, LC pressure sensor, and LC gas species sensor; and
a first receiving coil for receiving wireless signals from the LC temperature sensor, a second receiving coil for receiving wireless signals from the LC pressure sensor, and a third receiving coil for receiving wireless signals from the LC gas species sensor.

2. The LC-resonance sensor system of claim 1, wherein the transmitting coil is injected with a high-current pulse to generate a time-varying magnetic field that induces a voltage at the inductors of the LC temperature sensor, the LC pressure sensor, and the LC gas species sensor.

3. The LC-resonance sensor system of claim 2, wherein the time-varying magnetic field that induces a voltage at the inductors of the LC temperature sensor, the LC pressure sensor, and the LC gas species sensor induces a voltage at the first, second, and third receiver coils.

4. The LC-resonance sensor system of claim 1, wherein the LC temperature sensor, the LC pressure sensor, and the LC gas species sensor are oriented internal to an enclosure, and the transmitting coil and receiving coils are oriented external to the enclosure.

5. The LC-resonance sensor system of claim 1, wherein the LC temperature inductor further comprises a cylindrical inductor with coil windings with resistance that is sensitive to temperature.

6. The LC-resonance sensor system of claim 1, wherein the LC pressure sensor includes a piezo crystal bonded to a circular diaphragm forming an acoustic diaphragm which mechanically self-resonates at a controlled frequency.

7. The LC-resonance sensor system of claim 1, wherein the LC gas species sensor comprises a piezo-diaphragm at one end of cylindrical tube with the other end sealed off by a reflector plate.

8. The LC-resonance sensor system of claim 1, wherein the LC gas species sensor further comprises a piezo crystal bonded to a circular diaphragm attached to one end of the cylindrical tube with the other end of the tube closed using the reflector plate, and wherein the cylindrical tube comprises a plurality of holes to enable an exchange of gases.

9. The LC-resonance sensor system of claim 1, further comprising a computer processor with memory operationally connected to the LC-resonance system.

10. The LC-resonance sensor system of claim 10, further comprising a first signal processing algorithm, located within memory of the computer processor, which estimates the temperature inside an enclosure using the data recorded at the first receiving coil, a second signal processing algorithm that estimates the pressure inside an enclosure using the data recorded at the second receiving coil, and a third signal processing algorithm that estimates the gas species inside an enclosure using the data recorded at the third receiving coil.

11. The LC-resonance sensor system of claim 10, wherein the temperature is tracked by tracking the peak to peak to voltage level of the signal from the LC temperature sensor.

12. The LC-resonance sensor system of claim 10, wherein the pressure is tracked by tracking the shift in resonance peaks in frequency domain representation of the signal from the LC pressure sensor.

13. The LC-resonance sensor system of claim 10, wherein the gas species content is tracked by tracking the shift in resonance peaks in frequency domain representation of the signal from the LC gas species sensor.
